(19) United States
(12) Patent Application Publication
(10) Pub. No.: US 2003/0118553 A1
(43) Pub. Date: Jun. 26, 2003

(54) Vector constructs for gene-therapy mediated radionuclide therapy of undifferentiated and medullary thyroid carcinomas and non-thyroidal tumours and metastases mediated thereof

(76) Inventors: Thorsten Petrich, Hannover (DE); Eyck Potter, Hannover (DE)

Correspondence Address: Steven L Highlander, Fulbright & Jaworski, 600 Congress Avenue Suite 2400, Austin, TX, 78701, US

(21) Appl. No.: 10/182,536

(22) PCT Filed: Jan. 29, 2001
(86) PCT No.: PCT/DE01/00372

(30) Foreign Application Priority Data
Jan. 28, 2000 (DE) 100 03 653.8

(51) Int. Cl. A61K 48/00; C12N 7/00; A61K 9/127; C12N 15/861; C12N 15/867; C12N 15/88

(52) U.S. Cl. 424/93.2; 424/450; 435/456; 435/458; 435/235.1

(57) ABSTRACT

The invention relates to vector constructs, comprising vector DNA which includes regulatory sequences, the NIS gene coding for the sodium/iodide symporter, the TPO gene coding for thyroidal peroxidase and use thereof for production of a medicament/diagnostic for treatment/diagnosis of tumour disease states, whereby treatment occurs before or concurrent with a radionuclide therapy, in particular, with iodine-131, or astatine-211. The invention further relates to use of two or several vector constructs for production of a medicament/diagnostic for treatment/diagnosis of tumour disease states.

The present invention relates to vector constructs which are to be employed, assisted by liposomes or viruses as vectors, for the gene therapy of tumor diseases. In these vector constructs the genes for iodination (uptake of iodide/radionuclide) and iodization (metabolism of. iodide/radionuclide) of thyreocytes as well as tumor-specific promoters for the regulation of the gene expression are contained as cDNA.

Variants of such vector constructs containing the gene for iodination and a MCS (multiple cloning site) are furthermore suitable as reporter genes via the induced radionuclide uptake in target cells in vitro and in vivo (e.g., in cell culture and animal model). In the following these variants are referred to as reporter vectors. By means of a reporter vector containing an expression cassette to mediate the uptake of iodide the transfection efficiency of a transfection method may be determined quantitatively only by transfection and subsequent measurement of the uptake of radionuclide into the target cell and visualized locally via scintillation scanning or autoradiography. In case this reporter vector additionally contains an expression cassette of an arbitrary further gene, one may derive the expression level and the localization of the gene to be investigated via radionuclide scintillation scanning.

On the other hand, variants of the reporter vectors containing an expression cassette to mediate the uptake of radionuclide without promoter are suitable to determine promoter activities of any DNA fragments, likewise by measurement of the uptake of radionuclide.

Tumor diseases still belong to the most frequent causes of death, in spite of modem surgery, radiation therapy, and chemotherapy.

For a very rare tumor disease, the differentiated thyroid carcinoma, the radioactive iodine therapy following surgery of the thyroid gland is the most important therapy. This therapy makes use of the property of the tumor cells of the thyroid gland to store iodide (in particular radioactive I-131) in order to treat residual tumors and metastases by way of an “internal radiation therapy”. For surrounding normal tissue that does not store iodide it is a gentle therapy.

The radioactive iodine therapy has not been performed as yet by any type of cancer other than the differentiated thyroid carcinoma because only thyroid carcinomas due to proteins essential for biosynthesis of thyroid hormones (amongst them transport proteins, peroxidases, storage proteins) are capable to accumulate iodide and to store it in bound form over extended periods of time.

Therapy with I-131

According to the WHO (World Health Organization) cancer diseases were the second most frequent cause of death in the industrialized world in 1996. In many cases, classical tumor therapy consisting of surgery, radiation, and chemotherapy does not result in a satisfactory or permanent tumor remission.

A particular form of the radiation therapy is the so-called radioactive iodine therapy (RIT) based on the use of the radio isotope 1-131 in the form of iodide emitting β-particles, which therapy is employed only for differentiated thyroid carcinomas. The RIT has been employed successfully in the past 50 years and is based on the fact that the thyroid gland is the only organ of the human body exhibiting an active uptake of iodide (iodination) and further metabolizing the iodide to produce hormones of the thyroid gland (iodization) by binding the iodine to the tyrosine residues of an intracellular glycoprotein. The capacity to store iodide and thus the usefulness of therapy by means of RIT may be lost only if the tumor cells dedifferentiate if induced by RIT.

If, however, the capacity to store iodide is maintained over an extended period of time, I-131 may be reiteratively administered in several fractions at intervals of several months to treat metastases, a recidivation, or a residual tumor. The limiting factor of the RIT is the sensitivity of the blood-forming bone marrow to the ionizing radiation.

Even advanced tumor stages with extended lung or bone metastases are in many events curable by RIT. Other organ systems except the bone marrow will not be damaged deterministically which is in contrast to chemotherapy, external radiation, or surgery, which often cause deterministic damages.

In distinct cases therapy with I-131-MIBG (meta-iodo-benzyl-guanidine), like the radio iodine therapy with I-131 of thyroid carcinomas has been successfully performed for metastasizing pheochromocytomas (tumors of the adrenal medulla) and neuroblastomas. Antibodies (Ab) radioactively labeled with β emitters are presently being tested, e.g., for colorectal carcinomas (anti-CEA-Ab) or for B cell lymphomas (anti-CD22-Ab). However, the chances of curing these tumors have been comparatively low as yet.

Physiological Background

The iodide taken up in the thyroid follicles is first oxidized and incorporated into the tyrosine residues of thyreoglobulin (TG), a 660 kD glycoprotein, wherein mono-iodine and di-iodine tyrosine residues (MIT, DIT) are formed as intermediates. Approximately 80-90% of the iodide in the thyroid gland are bound in thyreoglobulin. Oxidation and transfer of iodide and the coupling of DIT residues are catalyzed by the thyroidal peroxidase (TPO) by means of H₂O₂, wherein thyroxin bound to TG is formed as a precursor of the proper thyroid hormone (T4). TG is taken up into the thyroid gland cells via endocytosis. Subsequently, T4 is released from TG by proteolysis. Thyroid-stimulating hormone (TSH) that is generated in the pituitary gland and commercially available also as a recombinant human hormone (rhTSH) mediates an enhanced uptake of iodide into the cytoplasm and an accelerated synthesis of TG through a stimulation of the membrane-bound TSH receptors of the thyreocytes via a second messenger (cAMP).

Molecular Background

Whereas the basic mechanism of the uptake of iodide into the thyreocytes has been unknown for a long time, the isolation and cloning of the NIS cDNA in the rat thyroid gland cell line FRTL-5 (Dai et al. 1996; WO 97/28175) first in 1996 and shortly thereafter in human thyreocytes (Smanik et al. 1996) were successful after a preliminary characterization of the sodium/iodide symporter (NIS) as a membrane-bound transport protein of the thyreocytes (MW 65 kD).

The expression of NIS in FRTL-5 cells is dependent upon TSH, as shown by Northern blot analysis. The gene expression commences after 3-6 h and reaches a maximum after 24 h. It could be shown by means of Western blot analysis that the representation of NIS protein on the cell surface of the FRTL-5 cells increases after 36 h and reaches a maximum after 72 h—parallel to the iodide transport activity—when stimulated with TSH.

Transfection of anaplastic thyroid cells in cell culture with NIS cDNA and a short-term iodide uptake were successful when vector pcDNA3 was used. The transfected tumor cells were subsequently implanted into rats and treated with I-131. It was found that the radioisotope had a relatively short intracellular life (effective half-life 6 h, maximum level after 90 min). A consequence thereof apparently was that due to the focal dose too low in the tumor no tumor reduction and thus no curing could be accomplished. Accordingly, this method may at best be used for diagnostic purposes. However, it cannot be used for in vivo therapy (Shimura et al. 1997).

A further conceivable therapy approach with iodine-labeled organic compounds (e.g., antibodies, oligonucleotides, receptor agonists) would be strongly suppressed by deiodases present in the peripheral blood, said deiodases having the capacity to cause an early demolition of such tracers, causing firstly a reduction of the effective half-life of the radiopharmaceuticals and secondly causing that other organs (e.g., liver, kidney, thyroid gland) are exposed to irradiation by radioactive degradation products.

Tumors other than differentiated thyroid carcinomas cannot as yet be therapeutically treated with radionuclides, as a sufficiently long half-life of the radionuclides in the tumor cells to accomplish a cytotoxic dose of irradiation is not warranted.

In summary, an oncological therapy by means of radionuclides has not yet been established for broad applications, except for the differentiated thyroid carcinoma.

Accordingly, the inventors have posed the object to develop a possibility to render available the radionuclide therapy to a number of different types of tumors. Thus, not only genes for the transport of iodide (WO 97/28175) or calcium (WO 98/45443) are to be transfected, which genes may possibly be responsible for an effective half-life of the radionuclides in the tumor cells which is too low, but genes for the binding (organification) of the radionuclides are to be co-transfected (transfected simultaneously) and on the same vector transfected, respectively, to improve the method. Furthermore, according to the invention even significantly more toxic emitters than only I-131(0.36 MeV; half-life: 8 d) are to be used for therapy. For this purpose, the present inventors evaluated the α emitter At-211 (5.8 MeV; half-life: 7.2 h) and other high-energy β emitters, e.g., Re-188 (2.1 MeV; half-life: 16.9 h) in vitro and in vivo.

That is, even dedifferentiated/anaplastic and medullar thyroid carcinomas (synonymous: C-cell carcinomas) without primary radionuclide storage and other non-thyroid tumors (e.g., of the kidney, of the mammary gland, of the prostate, of the stomach, of the lung, of the bone, of the pancreas, of the ovary, of the uterus, of the testis, of the brain, of endocrine and exocrine glands, of the skin, and of the intestine) that can presently not be treated by nuclear medicine and, in particular in a progressive stage, can hardly be treated in an effective manner, will be rendered available to a complementary or alternative therapy with radioactive iodide or other radionuclides.

These radionuclides are in particular astate (At-211) as an a emitter in the form of different, negatively charged species (e.g., At⁻, AtO⁻, AtO₂⁻, AtO₃⁻, AtO₄⁻), different, negatively charged species (in particular anionic oxygen compounds) of β emitters, e.g., of rhenium (Re-188 and Re-186) or yttrium (Y-90) but also Auger emitters such as gallium (Ga-67), indium (In-111), and iodine (I-123).

Not only solid tumors but also micrometastases or tumor cells circulating in the blood stream are to be treated by the gene therapy described according to the present invention. The therapy of micrometastases is in many cases crucial for the prognosis of tumor diseases, as a general tumor spreading in the body occurs via a micro metastasis. Detection of micrometastases by means of radiological methods is only very limited. Such micrometastases cannot be cured by a locally limited therapy of larger tumor masses (e.g., surgery, external irradiation).

The present inventors have provided vector constructs for a new method. By means of these constructs the property for uptake and storage of distinct anionic radionuclides (astate, rhenium, technetium, iodide etc.) into thyroid and non-thyroid human tumor cells for the purpose of diagnosis and therapy is transferred.

An aspect of the present invention thus relates to vector constructs as described in the claims. A further aspect of the present invention relates to the use of these constructs for the preparation of a diagnostic and therapeutic agent for the diagnosis and therapy of tumors, as described above, that is, in particular of tumor diseases such as dedifferentiated thyroid carcinomas, C cell carcinomas, non-thyroid tumors, and their metastases. For this purpose, the vector constructs of the present invention are employed along with the conventional RIT or other radionuclide therapies, as explained in detail in the claims. Particularly suitable are the constructs of the present invention also to check on the local radionuclide storage before the therapy and to detect as yet unknown tumor foci.

Variants of the vectors according to the present invention exhibiting only the NIS gene and an MCS (multiple cloning site) but not the TPO gene are suitable for further applications, namely as a reporter gene in vitro (cell culture) for the integration of any gene for the quantitative determination of the transfection efficiency via the uptake of radioactive iodine isotopes such as I-125. When using these vectors, it is additionally feasible to localize successfully transfected cells in vivo by means of a scintiscanning, e.g., I-123, I-131, Tc-99mO₄⁻ positron-emission tomography (PET) I-124, or autoradiography I-125. By means of this method the successfully transfected cells can, e.g., when transfecting the insulin gene or the dystrophin gene (or the respective cDNA) in co-transfection with the NIS gene in a gene therapy be visualized and localized in the body. These vector variants are defined in more detail in the claims, in particular in claim 13.

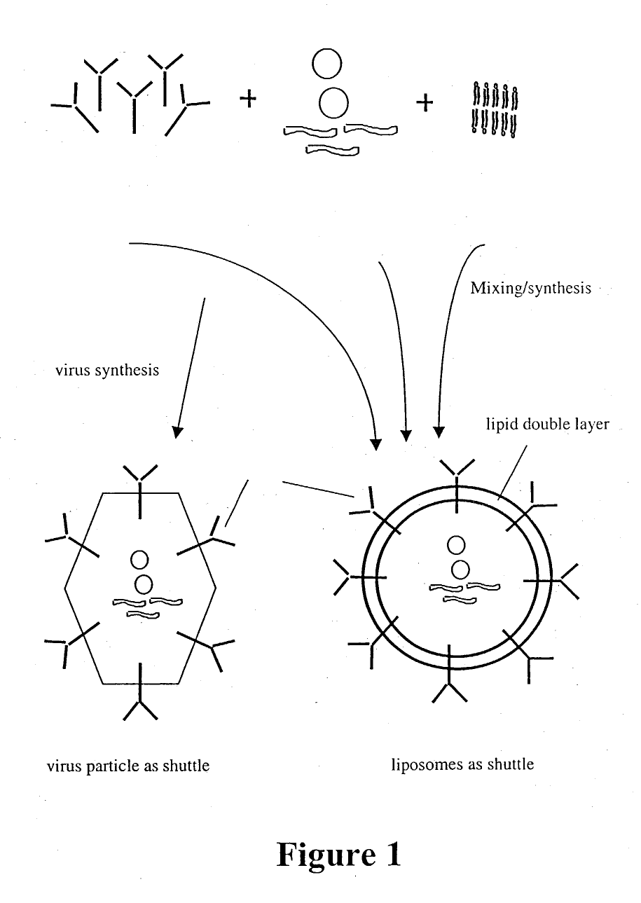

By a selective “shuttle-system” (liposomes or viruses with tumor affinity having the corresponding surface structures) the vector constructs of the present invention enter into the tumor cells. In other words, the NIS and TPO genes and optionally other relevant genes for the synthesis of the thyroid hormone (e.g., the TG gene) are (co-) transfected into these cells and expressed by means of, e.g., tissue-specific promoters. Within this method, the NIS gene mediates the cellular uptake of the radionuclides which, in turn, are bound via TPO to cellular substrates or TG or TG fragments. The binding to cellular proteins includes an extension of the cellular half-life of the radionuclides and increases the doses of irradiation (FIGS. 1, 2, and 3).

By these means, even dedifferentiated and medullar thyroid carcinomas and non-thyroid tumors can be treated due to the uptake and storage of the radionuclide.

The application of the radionuclides is, in case of I-131, in form of commercially available Na[I-131]I (capsules or solution for injection). Rhenium and yttrium isotopes (such as Re-188 and Y-90) are likewise commercially available and can be applied intravenously.

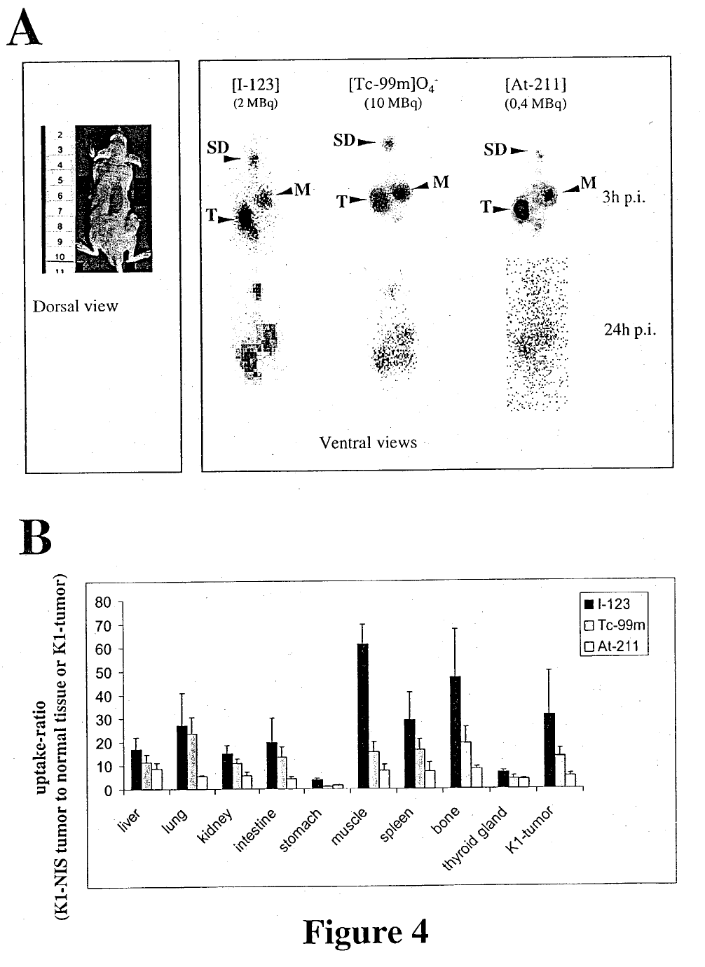

Quite conversely, astate is no naturally occurring element. At-211 can thus be produced in the cyclotron, utilizing the [²⁰⁹Bi(α,2n)²¹¹ At] reaction and bombardment of natural bismuth targets with α-particles (24.5 MeV) with a radiation current of 6 μA. The astate At-211 as used here was generated in the cyclotron (MC35scx, Scanditronix) and by dry distillation at 650° C. heated out of the target and recovered in a concentrated form in 0.02 M NA₂SO₃. This At-211 solution can be used in vitro and in vivo (animal model) for the diagnosis (81 keV γ-energy for scintiscanning) and therapy, once it has been diluted (see FIG. 4).

FIG. 1 schematically depicts the transfer of the genetic material. Monoclonal antibodies (or other cell-specific proteins such as surface antigens and receptor ligands) (Y-shape) and expression vectors (circles) with cDNA and regulatory sequences together with lipid solution are processed to liposomes as a shuttle system, wherein the monoclonal antibodies or the other cell-specific proteins are anchored in the membrane of the liposome, whereas the DNA is in the interior of the liposome. The monoclonal antibodies and the other proteins such as surface antigens and receptor ligands are to recognize the tumor cells and, as such, are favorable to enable the selective targeting of the desired cells (tumor cells) by the shuttle system. An alternative shuttle system is a virus particle (hexagon) in which the monoclonal antibodies and the other proteins are likewise anchored in the envelope of the virus, whereas the DNA is present in the interior of the virus.

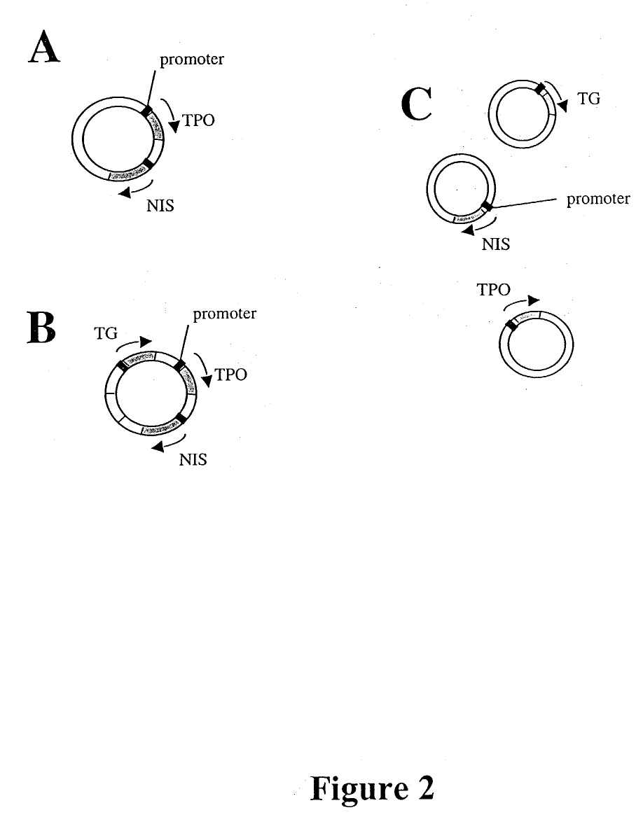

FIG. 2 schematically depicts the vector constructs of the present invention: A construct (A) with 2 genes (and 2 promoters), a construct (B) with 3 genes (and 3 promoters), or 3 constructs (C), each having one gene (and one promoter). Further DNA sequences (e.g., polyadenylation sites, regulatory sequences, origins of replication, or genes for selection) are not depicted, although they may be present or even must be present (such as the regulatory sequences).

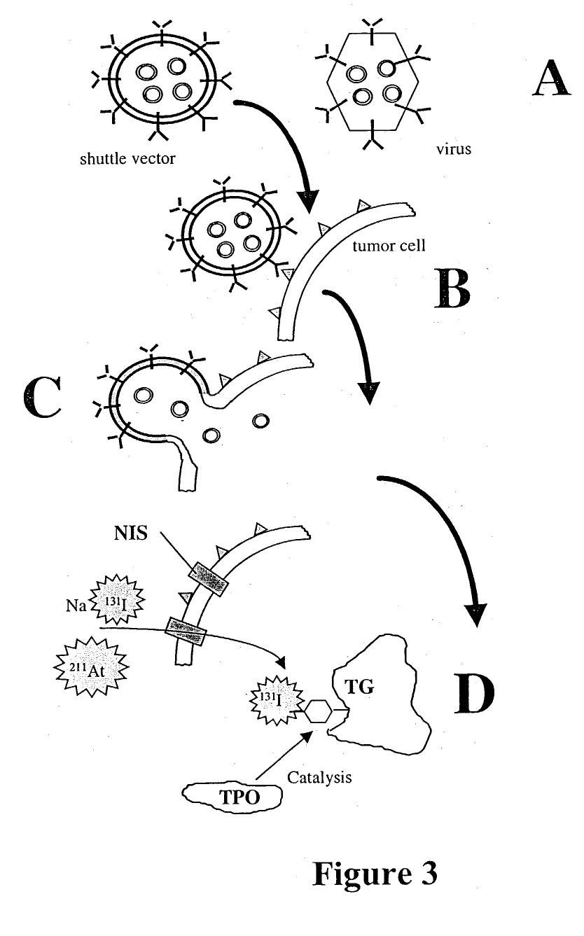

FIG. 3 schematically depicts the process of transfection. Liposome or virus particle (A) harboring the vector constructs of the present invention selectively or specifically dock to the membrane (with specific tumor antigens) of tumor cells, which is due to their surface structure (B), fuse with the cell membrane (C), and thereby enable the entry of the vector constructs (e.g., via endocytosis) into the tumor cell. The genes of the vector construct(s) will be expressed within the cell. As a consequence of the expression, in particular of the expression of NIS, the radionuclides will enter into the tumor cell and will be bound by TPO (D).

FIG. 4A depicts scintigraphic results of an animal model following subcutaneous injection of tumor cells of a thyroid carcinoma cell line (K1, ECACC #92030501) transfected with hNIS into the right flank of a NMRI nude mouse. Contra-laterally (left flank) the K1-wt control tumor can be recognized. After a tumor growth for 3 weeks scintigraphic pictures were taken ventrally by means of scintiscanning (gamma camera ZLC370, Siemens) 3 and 24 h following the injection of 2 MBq I-123, 10 MBq Tc-99m-pertechnetate and 0.4 MBq At-211 (RVL=right-ventral-left). Apart from the physiological enrichment of the tracer in the thyroid gland (SD) and stomach (M) a significant enrichment of all three tracers used can be seen in the tumor transfected with NIS, wherein the wild type control tumors (K1-wt) in the left flank are not shown.

In FIG. 4B the ratio of NIS tumor uptake of the three tracers (scintigraphically) and the uptake in normal tissue and K1-wt tumor of the three tracers is depicted. In the NIS tumor there is a 60-fold higher uptake of I-123, a 15-fold higher uptake of Tc-99m-pertechnetate, and a 10-fold higher uptake of At-211, as compared to muscle tissue.

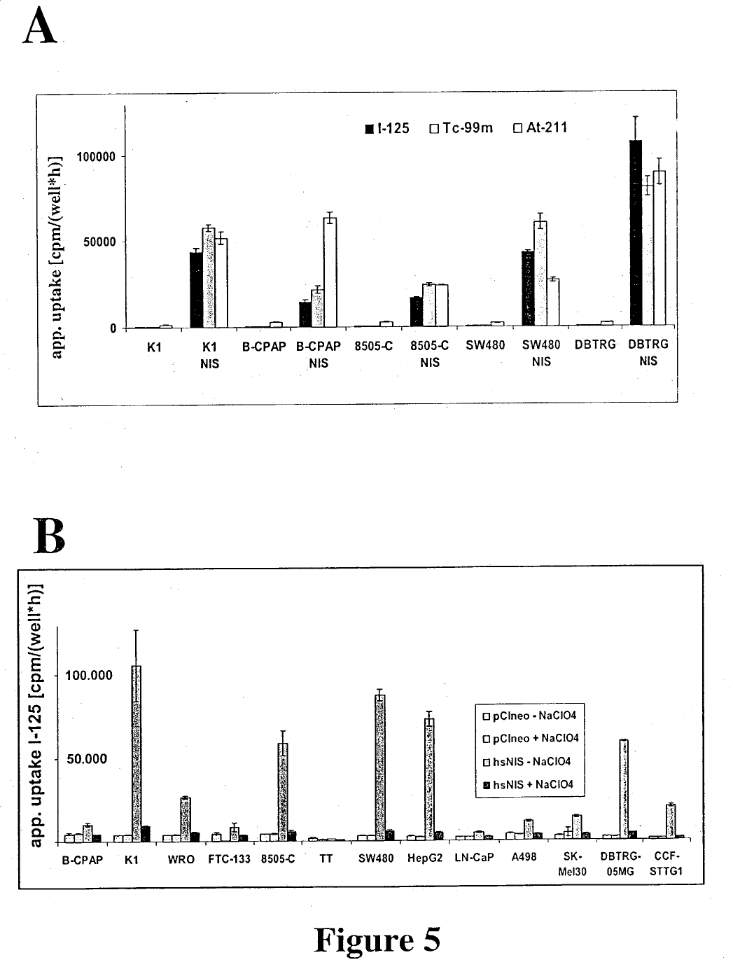

FIG. 5A depicts in vitro results of the radionuclide uptake of I-125, Tc-99m-pertechnetate, and At-211 in tumor cell lines stably transfected with the NIS gene as compared to the corresponding control cells: K1 papillary thyroid carcinoma ECACC #92030501, B-CPAP papillary thyroid carcinoma DSMZ #ACC273, 8505-C anaplastic thyroid carcinoma DSMZ #ACC219, SW480 colon carcinoma DSMZ #ACC313, DBTRG glioblastoma DSMZ #ACC359. There is a highly significant uptake of all nuclides including the astate only in tumor cell lines transfected with NIS, whereas there is no uptake in the control cells.

In FIG. 5B the uptake of I-125 is additionally shown depending on a perchlorate blockage in transiently transfected tumor cell lines. A significant iodide uptake was found not only in the above-mentioned cell lines but also in the following cell lines: Follicular thyroid carcinoma ECACC #92030502, HepG2 hepatocellular carcinoma DSMZ #ACC180, LN-Cap prostate carcinoma DSMZ #ACC256, A498 kidney cell carcinoma DSMZ #ACC55, SK-Mel30 malignant melanoma DSMZ #ACC151, CCF-STTG1 astrocytoma ECACC #90021502, TT medullar thyroid carcinoma ECACC #92050721. The differences in the iodide uptake rates in different cell lines transfected with NIS are caused by the variable transfection efficiency of the distinct cell lines with the method used.

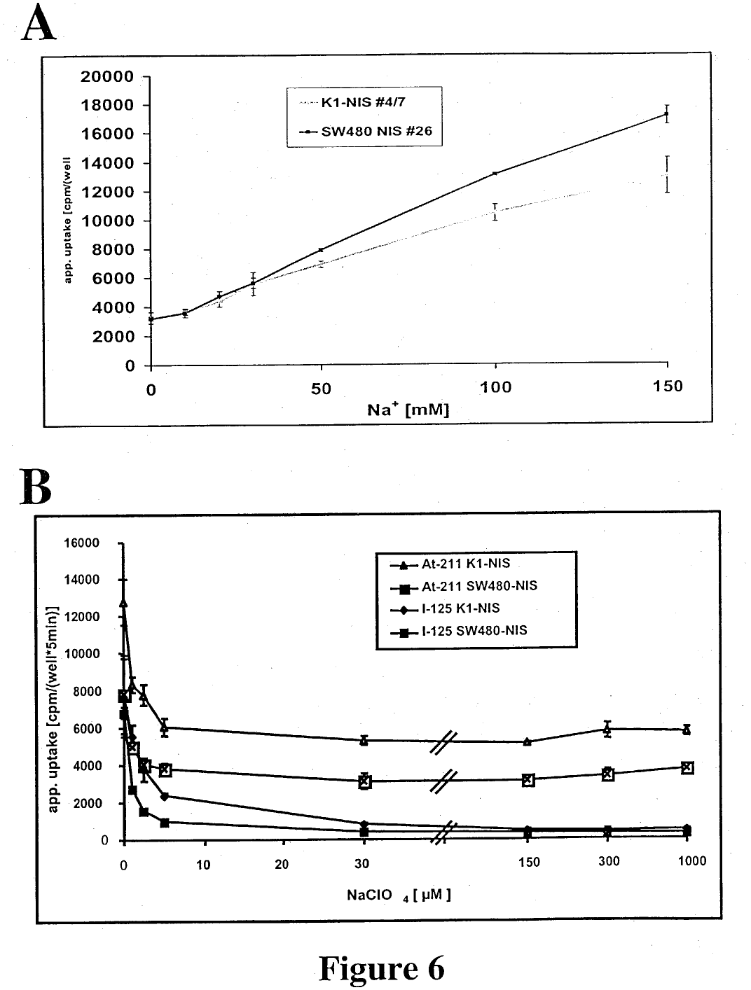

FIG. 6A depicts the dependency upon Na⁺ of the At-211 transport in vitro in stably transfected tumor cell lines (K1-NIS, SW480-NIS), wherein NaCl has been iso-osmotically replaced by choline chloride. There is a clear-cut dependency upon Na⁺ of the astate transport which is also known from the iodide. These results, as does the clear inhibition of the astate transport by small perchlorate concentrations (FIG. 6B), confirm the hypothesis that astate is transported via the NIS as is the iodide.

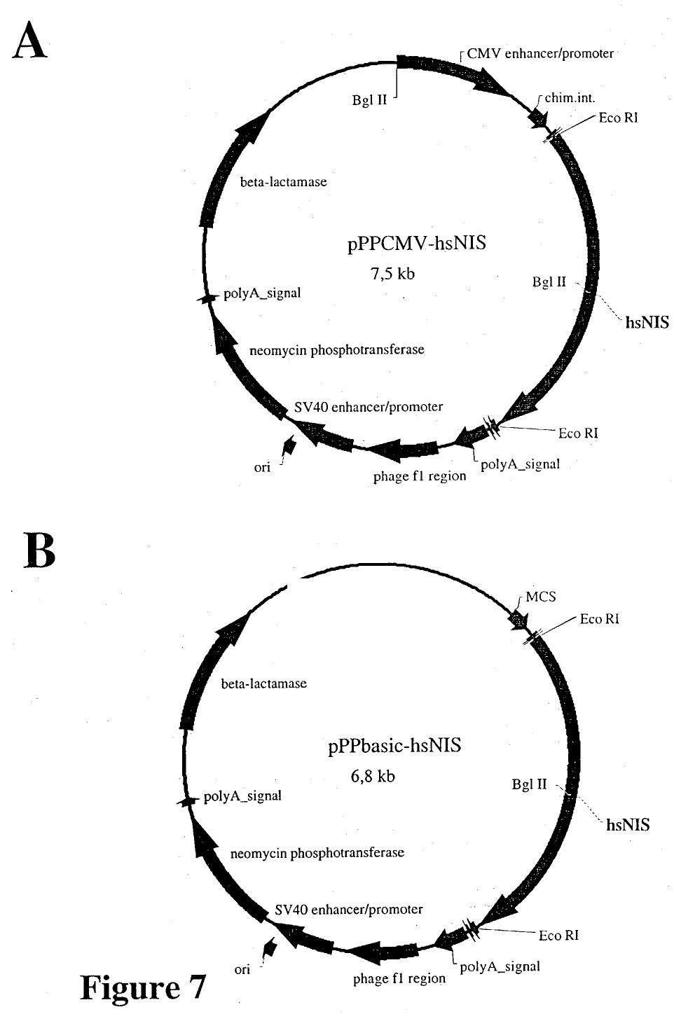

FIG. 7A depicts the vector pPPCMV-hcNIS derived from the vector pCIneo (Promega GmbH, Mannheim, FRG) that was used according to the invention for the transfection of the NIS gene into different tumor cells as well as for the in vivo experiments in nude mice. The human NIS gene (HNIS) is under the control of the CMV promoter. The CMV promoter can be replaced by tissue-specific promoters.

FIG. 7B depicts a variant of the vector of FIG. 7A, which variant is suitable to check on any promoter located in 5′-position, which promoter can be cloned into an MCS.

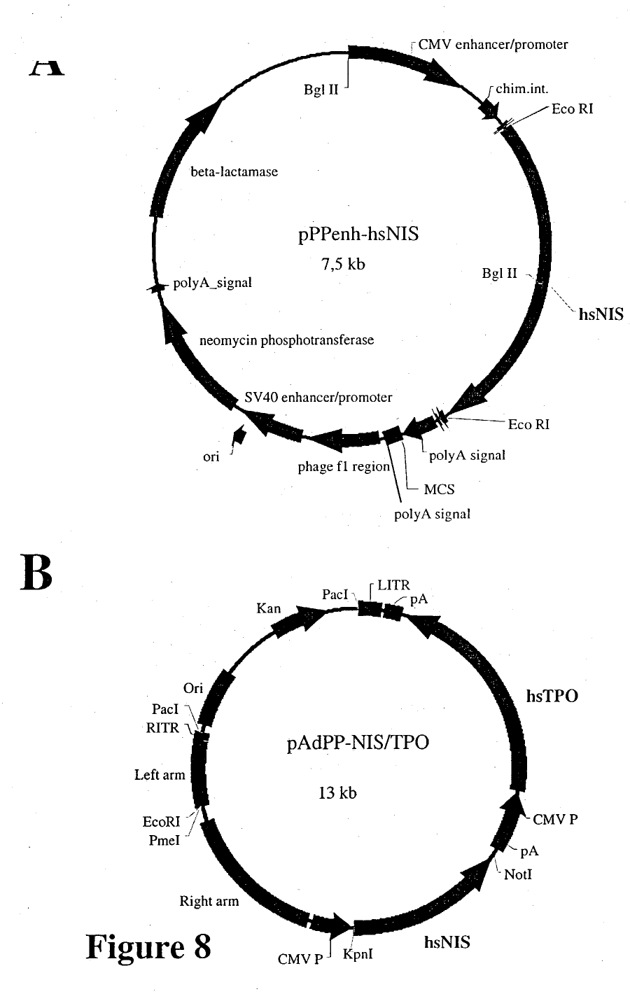

FIG. 8A depicts the vector pPPenh-hsNIS, a further variant of the vector pPPCMV-NIS as a reporter vector with an MCS located in a 3′-position to the NIS gene for the cloning of a desired gene. By measuring the iodide uptake the in vivo or in vitro expression of other genes can be localized or quantified and the transfection efficiency of a transfection method be investigated, respectively.

FIG. 8B depicts the Vector pAdPP-NIS/TPO which is derived from the shuttle vector pAdTrack-CMV (He et al. 1997) and is suitable to generate replication-deficient adenoviral vectors, which vectors can simultaneously introduce the HNIS gene and the human TPO gene into tumor cells and express both therein when under the control of the CMV promoter and a tissue-specific promoter, respectively.

According to a further preferred embodiment of the present invention a third gene, apart from the NIS and the TPO genes, is (co-) transfected: The TG gene or a TG gene fragment coding for a protein portion containing tyrosin residues functioning as iodide acceptors. The thyreoglobulin (TG) functions as an iodine or iodide storage in the cells (Malthiery et al., 1987).

Thus, as already done in the RIT of differentiated thyroid carcinomas, an extension of the effective half-life and the irradiation period of the tumor cell nucleus and, thus, the induction of a therapeutically effective dose (effective half-life in the classical RIT: 5 days) is induced. In case the (co-) transfection is done selectively into the tumor cells (e.g., by means of targeting by membrane epitopes or by tumor-specific promoters), damage of other organs by the irradiation is small (radiation load of the whole organism: about 300 mSv with a standard activity in the RIT of about 11.1 GBq) as is the case in the classical RIT. A bone marrow aplasia or a bone marrow depression can be avoided in cases when the targeted partial body dose of the bone marrow does not exceed 2 Gray [Gy]. Corresponding dose measurements are feasible during therapy and belong to the prior art.

The transfer of the genetic material into the tumor cells occurs via shuttle vectors. These shuttle vectors may be liposomes exhibiting membrane-bound monoclonal antibodies (Ab), ligands or proteins for the recognition of distinct tumor cell surface structures. These proteins or antibodies integrated into the liposome membrane are to perform a first selection within the tissue-specific targeting, thereby transporting the genes to be transfected with a higher degree of probability into tumor cells than in normal cells. Alternatively, tumor-specific, specifically prepared, replication-deficient viruses (e.g., adenoviruses, lentiviruses, retroviruses, other DNA viruses) exhibiting the corresponding epitopes may also be used: Adenoviral vectors effect a transient transfection, since the DNA does not integrate into the genome and, thus, is lost after several cell divisions, which is in contrast to the retroviruses. Adenoviruses do not need proliferating cells for infection and gene expression, which is a further contrast to the retroviruses, and which is an advantage in case of slowly growing or resting tumors. Retroviruses can only be used for dividing tumor cells, which will subsequently be permanently transfected. These reasons argue for a preferred employment of human or animal pathogenic (e.g., sheep) adenoviral vectors. Moreover, it is feasible to modify adenoviruses such that they may recognize binding sites typical of tumors. Adenoviruses have so-called fiber proteins with terminal “pips” (trimer, L-region IV “late expression”, 62 kD). By means of these fiber proteins the viruses bind to membrane-bound receptors (CAR, Coxsackie- and adenovirus-receptor) of their target cells and enter into the cytoplasm via endocytosis [Bergelson et al. 1997]. The structure of these pips is encoded by the virus DNA (L-region) and may be modified by changing the underlying sequence. If the sequence encoding the monoclonal antibody specific for distinct membrane epitopes (e.g., PSMA) is cloned into the sequence encoding the pips or replaces the sequence encoding the pips, the viruses can infect more cells expressing PSMA (prostate-specific membrane antigen) (e.g., prostate carcinoma). By these means, there is a first selection of the tumor cells in regard of a future therapy with radionuclides. As the pips are immunogenic in their original structure, a change of the structure into the direction of human tumor cell epitopes by modifying the virus genome can decrease the immunogenicity of the fiber proteins, thereby allowing a several-fold application to the patient and a decrease of the risk of allergies.

The generation of such virus particles or liposomes has already been described and belongs to the prior art (Strauss et al., 1997; Tarhovsky et al., 1998; Anderson, 1998; Martin et al., 1999; Kurane et al., 1998). A further possibility to transfer the therapeutic genes into tumor cells is the application of virus producing cells (e.g., psi 2-BAG packaging cell line) directly into the tumor in neighboring tissue, or elsewhere, e.g. into the muscles. The virus particles produced in the body by the implanted cells (“producer cells”) are directly in the tumor or nearby and can transform the tumor cell (Short et al. 1990). When using tumor-specific promoters in the vector constructs or tumor-specific surface structures of the virus envelopes (e.g., fiber proteins) in this type of application, the virus producing cells may be even used in a location in the body remote from the tumor, as the virus particles generated invade only tumor cells with corresponding epitopes, and the therapeutic genes are expressed only tumor-specifically.

Examples for tumor membrane epitopes are:

Mamma carcinoma: erbB2 (Slamon, 1987)

Ovary carcinoma: CA125, HMFG1 and HMFG2 (Metcalf et al. 1998),

Prostate carcinoma: PSMA (Murphy et al. 1998),

Malignant melanoma:Melan-A/MART-1-AG (Schneider et al. 1998).

The proper tumor specificity is to occur only in the tumor cells by way of a directed regulation of the gene expression. Therefore, in the vector constructs in front of the genes to be transfected specific promoters are placed allowing an expression of the proteins (NIS, TPO, TG, hereinafter designated as therapeutic proteins) only in specific tissues, depending on the type of the tumors. Suitable promoters are promoters of tumor-specific proteins. These proteins may be tumor markers (e.g., TG, CEA, calcitonin, AFP, CA-19-9, PSA), receptors (e.g., SMS receptor), membrane proteins (e.g., PSMA), enzymes (e.g., NSE), hormones (prolactin, HCG), or other peptides, which are produced by the tumor cells to a high extent but only by few body cells. Thus, a tumor-specific overexpression of the genes to be transfected is warranted under the control of distinct promoters, and the radionuclides are stored only in distinct tumor cells but not in normal tissue (even if this normal tissue is also transfected by viruses or liposomes). Tumor-specific promoters are known, have been sequenced, and may be considered for this type of therapy. The directed expression of genes under control of distinct promoters belongs to the prior art. For example, the following promoters are suitable for the corresponding tumor diseases:

erbB2-, Ca-15-3 promoter: mamma carcinoma

Calcitonin promoter: medullar thyroid carcinoma

CEA promoter: stomach tumor, intestine tumor, anaplastic thyroid carcinoma, bronchial carcinoma, ovary carcinoma

TG promoter: papillary and follicular thyroid carcinomas

NSE promoter: Small cell bronchial carcinoma

PSA, acid phosphotase promoter: prostate carcinoma

SMS receptor promoter: kidney cell carcinoma, medullar thyroid carcinoma, carcinoids, pituitary tumor

AFP promoter: uterus carcinoma, liver, ovary and testis tumor HCG, LDH promoter: ovary and testis tumor

SCC promoter: cervix carcinoma, lung tumor, epithelial

CA-19-9, Ca-50 promoter: colon, pancreas carcinoma

Ca-125 promoter: ovary tumors, epithelial tumors

ACTH promoter: bronchial, mamma, pancreas, stomach carcinoma

Prolactin promoter: pituitary tumors

In the vector constructs the therapeutic genes have to be combined individually with the corresponding promoters, depending on the tumor disease. According to a preferred embodiment the vector constructs are packaged into liposomes, the membranes of which bear antibodies or proteins against tumor cell surface structures such as PSMA-Ab, SMS analog, or erbB3-Ab in an integrated manner. The transfection of the therapeutic genes preferably occurs with a number of circular vectors corresponding to the number of therapeutic genes to be transfected. The circular vectors each exhibit (i) a promoter region (e.g., SV40-P_{E}, CMV promoter or tissue-specific promoter such as PSA or CEA promoter), (ii) an origin of replication functional in mammalian cells (e.g., Repori_{BAk.}/Repori_{Mam.}), (iii) an antibiotic resistance gene (e.g., amp^{r}, neo^{r}), (iv) a polyadenylation signal (e.g., base sequence: AATAAA) and (v) cDNA for one of the therapeutic proteins.

According to another preferred embodiment (FIG. 7A, example) the vector constructs are packaged also in liposomes, the membranes of which bear antibodies or proteins against tumor cell surface structures in an integrated manner. The transfection of the two or three therapeutic genes (NIS, TPO, TG) now occurs by means of a circular vector, however, the vectors comprising not only the above-mentioned DNA segments (i) to (iv) but also the cDNAs of two or three therapeutic genes (e.g., vector a) with NIS and TPO cDNA and vector b) with NIS, TPO, and TG cDNA).

A further preferred embodiment according to the present invention (FIG. 8B, example) uses viral vectors rather than liposomes as shuttle vectors. A preferred variant is the integration of the NIS and the TPO gene with a corresponding tumor-specific promoter region or a constitutive promoter (e.g., of CMV, SV40) into replication-deficient adenoviral genomes (e.g., pShuttle/Ad-easy-Kit, Quantum), the surface antigens of which can be supplemented with tumor-specifically modified surface proteins (e.g., against the PSMA antigen, the SMS receptor, the erbB3-antigen). Likewise, all three genes (NIS, TPO, TG) may be integrated into one or separately into three different viral vectors with the corresponding tumor-specific promoter region or in different retroviral vectors with distinct tumor-specific surface proteins (e.g., against PSMA antigen, SMS receptor, erbB3 antigen).

Suitable vector constructs are circularly closed, non-restrictive expression vectors, e.g., simian virus 40 (SV40) or a pSV2 type derived therefrom, but also derivatives of adenovirus, retrovirus, or herpes virus genomes exhibiting a transcription rate in the eukaryotic nucleus as high as possible. The object is the expression of the therapeutic genes and the representation of the corresponding “novel” thyroid proteins on the cell membrane and in the cytoplasm of the tumor cells. The preparation of the vector constructs and the cloning into the virus genomes belong to the prior art (see also Sambrook et al.: Molecular Cloning).

The therapeutic application of the liposomes or of the viruses being pathogenic for humans or animals or cells producing such viruses occurs via the intravenous, the intraperitoneal, the intrathecal, the intracranial, the intrathoracal, the endobronchial, the endolymphatic, the intraarterial, or the intratumoral route (in case of the application of the cells: Intratumoral route is preferred). When the application is intravenous and systemic, it is preferred to use tumor-specific promoters in the vector constructs. When the application is direct within a local therapy into the tumor (e.g., intratumoral or into an artery providing the tumor), constitutive promoters without tumor specificity (e.g., CMV or SV40 promoter) may be used.

Prior to a therapy one should take care that the tumor marker values or the expression of the receptors or of the membrane-bound proteins or other tumor-specific proteins is sufficiently high. This can be checked, e.g., in a blood sample or by a biopsy from the tumor tissue.

Cloning

The synthesis of the cDNAs (of the therapeutic genes) belongs to the prior art. Likewise, the preparation of the circular (or linear) vector constructs with the corresponding regulatory elements and restriction cleavage sites to insert the cDNAs is also known. In a preferred embodiment each cDNA of a vector construct has its own efficient promoter region, e.g., the promoter region of CMV (immediate early promoter/enhancer) or of the SV40 virus (SV40-P_{E}), that of the Rous Sarcoma virus (RSV-LTR), or that of the thymidine kinase gene of the herpex simplex virus (HSV-tk). Additionally, several therapeutic genes may be regulated by one common promoter. In such case, at least two of the therapeutic genes must be localized on a single vector molecule.

The cDNAs of the therapeutic genes are amplified from human thyreocyte mRNA after reverse transcription by subsequent PCR with gene-specific primers, and the fragments thus obtained are subcloned in a cloning vector (e.g., pCRblunt, Invitrogen) in E. coli (K12 derivative). For this purpose, overlapping cDNA segments of the single genes are used, the fragments allowing the generation of full-length cDNAs due to their common restriction enzyme cleavage sites. The cDNAs are cloned into a vector for constitutive expression in mammalian cells (e.g., in pCIneo under control of the CMV promoter, Promega). The expression vectors with integrated cDNA (FIG. 7A, example) are used for transient transfection of different tumor cell lines (Fugen6, Roche) and for the generation of stably transfected cell lines by antibiotic selection (e.g., G418 for pCIneo). Radionuclide uptake measurements ([I-125], [At-211], [Tc-99m], [Re-188]) with cell cultures (FIGS. 5 and 6) are performed according to the method of Weiss et al. 1984. To establish an animal model stably transfected tumor cell lines and the corresponding control cells, respectively, are subcutaneously injected into nude NMRI mice (nu/nu). After tumor growth in the course of 3-4 weeks and application of different radionuclides (i. a., [I-123], [At-211], [Tc-99m]) sequential scintiscans are taken with a gamma camera and organ distribution over region of interest (ROI) analyses determined (FIG. 4).

The results of the experiments in vitro and in vivo (animal model) demonstrate that a significantly higher radionuclide uptake can be induced in tumor cells by the transfection of the therapeutic genes as described, as compared to the uptake in the normal tissue or in the control tumors. This particularly applies to astate but has not yet been published. The proteins expressed by means of the vectors of the present invention during transfection are detected in a Western blot with specific antibodies. An organification of the radionuclides by means of the vectors is detected by comparing studies with NIS transfectants and control cells by measuring the ethanol pellets. The organification of the radionuclides which is crucial for the extension of the effective half-life can be obtained by the additional use of TPO. In efflux experiments extremely short half-lives are measured for the transfection with the NIS gene alone (half-life: 5 min), which measurements occur by a change of the medium of the cell culture dishes after an incubation with different radionuclides. The co-transfection of NIS, and TPO, and of NIS, TPO, and TG, respectively, effects a significant extension of the binding of the radionuclide by the factor of 7-10 in vitro and, thus, an increase of the radiation dose in the transfected tumor cells, however. Apoptosis rates are compared on clonogenic assays after transfection with NIS alone as compared to a co-transfection with NIS and TPO after incubation with α-β emitting radionuclides. It is shown that the apoptosis rate in case of the co-transfection of both genes is increased by the factor of 20-30 as compared to a NIS-transfection alone, both for tumor cells derived from the thyroid gland and for tumor cells from other tissues. In addition to the Western blot analysis to detect the proteins (NIS, TPO, TG) the enzyme (peroxidase) activity of TPO is measured in parallel approaches in the guaiacol assay.

Vector variants to determine the transfection efficiency, the promoter activity, and the visual illustration of a transfection are determined by co-transfection of LN-CaP cells (prostate carcinoma, DSMZ #ACC-256) with the vectors pPPbasic-hsNIS (CMV and PSA promoter vs. without promoter) and pPPenh-hsNIS with pSV-βGal and pEGFP (Promega).

Body cells which may take up the vector constructs in a therapy “en passant” do not possess the relevant transcription factors necessary to switch on the promoter-dependent expression. Therefore, these cells cannot express the transfected genes, they cannot store the radioisotopes, and will therefore remain essentially undamaged. Accordingly, by individually selecting a suitable promoter an additional safety of the therapy can be obtained.

A therapy of distinct tumor cells not successfully transfected is also accomplished by irradiation from neighboring tumor cells that have been transfected. Electrons of β emitters of the energy of 0.61 MeV (I-131) have a range of 2 mm (average range: 1 mm) in water/tissue, said range corresponding to about 40 cell layers. Significantly greater ranges in the tissues are known for Re-188 and Y-90. α-particles originating during decomposition of At-211 have comparatively much higher energy (>5 MeV) and due to their high mass a very much higher ionization density, explaining their higher toxicity. One may assume that only one α-decomposition in the interior of a cell is sufficient to kill it. Apart from the ionization, recoil/reaction forces developing during decomposition and resulting in significant devastation of the intracellular matrix also play a role.

For this reason the object of the present invention includes to import into the tumor cells in particular At-211 and to fix it therein, where it does the greatest harm.

Prior to a transfection the receptor status and the status of distinct membrane epitopes of the tumor, respectively, is determined in order to ascertain an efficient passing-in (entry) of the therapeutic genes. This may occur by means of an immunocytology, immunohistology, or immunoscintiscanning.

Additionally, a block of the iodide and radionuclide, respectively, uptake of the thyroid gland along with a short-term high dosage of thyroid hormones (e.g., 1 mg/d L-thyroxin over a period of 3 days) for the suppression of the pituitary TSH production is required in order to avoid that greater amounts of the radionuclides are trapped. In extreme cases of a hyperthyroidism prior to a tumor treatment optionally a thyroidectomy has to occur, because physiologically radioiodine or other radionuclides are preferably taken up by the thyroid gland. Consequently, they are no longer available for the incorporation into the tumor cells. In such cases a substitution with thyroid hormone may be performed post-therapeutically (after a thyroidectomy).

At the onset of the proper treatment, as described herein, shuttle vectors carrying the therapeutic genes (NIS, TPO, TG) and viral vectors as described above, respectively, are generated in vitro and, e.g., intravenously administered in a carrier solution (FIG. 1).

After an interval of about 2-4 days the therapeutic genes have passed in the tumor cells. The synthesis of the therapeutic proteins (NIS, TPO, TG) occurs after transcription of the cDNA in the cell nucleus by translation of the mRNA in the tumor cytoplasm. At that point of time a radionuclide whole body scintiscanning (e.g., trial examination with a low activity of about 50-100 MBq I-131) is thus to be performed in order to intratumorally check for the storage capacity. Depending on the acquired storage capacity of the tumor tissue, the hospitalized patient is subjected to a high dose radionuclide therapy (with, e.g., 4-12 GBq I-131).

For the reason of radiation protection, therapy must be performed in a ward of nuclear medicine furnished for the handling of open radionuclides (shielding, decay facilities). The combination of transfection and radionuclide therapy can be repeated several times, provided there is good tolerance. In case an immunization against the viruses or liposomes results, the “transport vehicle” must be changed or a short-term immunosuppressive therapy be initiated. A small immunosuppression in spite of an infection with viruses is possible, because the injected viruses are replication-deficient and cannot replicate in the body. Similar to the classical RIT, in terms of radiation protection the maximum of 4-5 fractions annually is recommended. Preferred, however, is an only transient transfection (3-4 weeks) by means of adenoviral vectors, since the expression of the therapeutic genes for the radionuclide therapy is only required for short periods and has not to occur permanently.

The gene therapy according to the present invention enables no more than a transient transcription and overexpression (exception: when using retroviruses as a shuttle system) that is maintained possibly for few cell divisions but which is sufficient for the purpose of subsequent radionuclide therapy with a transient storage and subsequent apoptosis of the tumor cells.

The use according to the present invention of the vector constructs described in detail above, which vector constructs comprise the genes responsible for the radionuclide uptake and the organification, provides the following crucial advantages:

Dedifferentiated and medullar thyroid and C cell carcinomas are provided with the property to take up and store radionuclides.

Even tumors other than the differentiated thyroid carcinomas are treatable due to a gene technological modification to store radionuclides for longer time periods; this renders these tumors treatable by nuclear medicine.

The storage of radionuclides occurs selectively only in transfected cells, all other organs do not store the radionuclides which is tantamount with a gentle treatment.

There is no damaging of the surrounding tissue, because the range of the α/β radiation in the tissue is small; essentially, the radiation destroys the DNA of the storing tumor cells and the cells immediately surrounding them, respectively. Thus, the term of the treatment is not limited (bone marrow excluded), which is in contrast to the term of treatment in case of external irradiation.

If only few of the tumor cells are successfully transfected, neighboring non-transfected tumor cells may be damaged as well by an irradiation from the neighborhood. In such case, radionuclides may additionally enter into neighboring non-transfected tumor cells via cell pores (gap junctions) and damage them.

The radiation dose intratumorally is significantly higher for the radionuclide therapy (>300Gy) than for the external irradiation (about 40-60Gy). Thus, the toxicity and therapeutic effect in the tumor is stronger and the local side effects to be expected are smaller.

Even if only a transient radionuclide storage takes place via transient gene expression, this effect is sufficient to perform therapy.

As prior to the onset of the radionuclide therapy the receptor/membrane status of the tumor is determined and liposomes and viruses, respectively, may be prepared accordingly, the risk of side effects is small.

By selecting upstream tumor-specific promoters, that is, tumor-specific promoters localized in 5′-position, depending on the tumor marker detection or protein synthesis of the tumor for the regulation of the gene expression, a high selectivity of the tumor treatment can be accomplished.

The transfected tumor cells are detectable via a scintiscanning with gamma cameras (or in greater exactness by means of positron-emission tomography with the positron emitter [I-124]) for the staging (distribution diagnostics) or for a control of the course after therapy.

The damage of the bone marrow is the limiting factor in case of the radionuclide therapy. Improvement thereof may be achieved, however, by an autologous bone marrow transplantation or stem cell support.

As radionuclide therapies are internationally established for the treatment of thyroid cancer, therapy wards in hospitals specialized in nuclear medicine exist already today world-wide; in such hospitals the therapy according to the present invention may be performed.

Next to the therapy with I-131 in particular also α emitters (e.g., At-211) may be employed in case of different tumor diseases, said emitters exhibiting an extremely high local toxicity.

### REFERENCES

Anderson W F: Human gene therapy. Nature 392 (6679 Suppl): 25-30, 1998.

Bergelson J M, Cunningham J A, Drouguett G et al: Isolation of a common receptor for coxsackie B viruses and adenoviruses 2 and 5. Science 275: 1320-1323, 1997.

Dai G et al: Cloning and characterization of the thyroid iodide transporter. Nature 379: 458-460, London 1996.

He T C, Zhou S, Da Costa L T, Yu J, Kinzler K W, Vogelstein B: A simplified system for generating recombinant adenoviruses. Proc Natl Acad Sci USA 95 (5), 2509-5514, 1998.

Kurane S, Krauss J C, Watari E, Kannagi R, Chang A E, Kudoh S: Targeted gene transfer for adenocarcinoma using a combination of tumor-specific antibody and tissue-specific promoter. Jpn J Cancer Res 89(11):1212-9, 1998.

Malthiery Y, Lissitzky S: Primary structure of human thyroglobulin deduced from the sequence of ist 8448-base complementary DNA. Eur J Biochem 165 (3), 491-498, 1987.

Martin F, Neil S, Kupsch J, Maurice M, Cosset F, Collins M: Retrovirus targeting by tropism restriction to melanoma cells. J Virol 73(8);6923-9; 1999.

Metcalf K S et al: Culture of ascitic ovarian cancer cells as a clinically-relevant ex vivo model for the assessment of biological therapies. J Eur Gynaecol Oncolo 19: 113-119, 1998.

Murphy G P et al: Measurement of serum prostate-specific membrane antigen, a new prognostic marker for prostate cancer. J Urology 51: 89-97, 1998.

Schneider J et al: Overlapping peptides of melanocyte differentiation antigen Melan-A/MART-recognized by autologous cytolytic T-lymphocytes in association with HLA-B45.1 and HLA-A2.1. J Cancer 75: 451-458, 1998.

Shimura et al: Iodide Uptake and experimental ¹³¹I Therapy in transplanted undifferentiated thyroid cancer cells expressing the Na+/I-symporter gene. Endocrinology 138: 4493-4496, 1997

Short M P, Choi B C, Lee J K, Malick A, Breakefield X O, Martuza R L: Gene delivery to glioma cells in rat brain by grafting of a retrovirus packaging cell line. J Neurosci Res 27 (3): 427-39, 1990.

Slamon D J: Proto-oncogenes and human cancers. In: New England J Med 317: 955-957, 1987.

Smanik P A, Liu Q, Furminger T L, Ryu K, Xing S, Mazzaferri E L, Jhiang S M: Cloning of the human sodium iodide symporter. Biochem Biophys Res Commun 226(2):339-45, 1996.

Strauss M, Barranger J A: Concepts in gene therapy. Walter de Gruyter, Berlin New York, 1997.

Tarhovsky Y S, Ivanitsky G R: Liposomes in gene therapy. Structural polymorphism of lipids and effectiveness of gene delivery. Biochemistry 63 (6): 607-18, 1998.

Weiss S J, Philp N J, Grollman E F: Iodide transport in a continuous line of cultured cells from rat thyroid. Endocrinology 1984;114(4):1090-8.

1. Vector construct, comprising vector DNA including regulatory sequences as well as the NIS gene encoding the sodium/iodide symporter and the TPO gene encoding the thyroid peroxidase.

2. The vector construct of claim 1, characterized in that it further comprises the TG gene encoding the human thyreoglobulin or a nucleic acid sequence encoding a physiologically active fragment of human thyreoglobulin.

3. The vector construct of any of the preceding claims, characterized in that it comprises the SV40-PE, CMV, or a tissue-specific promoter.

4. The vector construct of claim 3, characterized in that the tissue-specific promoter is the promoter of a gene of a tumor-specific protein, of a tumor-specific enzyme, of a tumor-specific receptor, of a tumor-specific membrane component, or of a tumor-specific tumor marker.

5. The vector construct of any of the preceding claims, characterized in that it further comprises an origin of replication and/or a marker gene, e.g., an antibiotic resistance gene, and/or a polyadenylation signal.

6. The vector construct of any of the preceding claims, further comprising a multiple cloning site (MCS), preferably in a position in 5′ direction of the NIS gene.

7. The vector construct of any of claims 1 to 5, further comprising a multiple cloning site (MCS), preferably in a position in 3′ direction of the NIS gene.

8. The vector construct of any of the preceding claims, characterized in that it is included in a liposome, wherein the liposomes may exhibit membrane-bound antibodies, in particular monoclonal antibodies, or other proteins, in particular receptor ligands, specific for tumor surface structures.

9. The vector construct of any of claims 1 to 7, characterized in that it is included in recombinant adenoviral, retroviral or other viruses of human or animal pathogenicity, wherein the viruses may exhibit on their surface natural or artificial tissue-specific membrane-bound proteins (e.g., fiber proteins) or receptor ligands specific for tumor surface structures.

10. The vector construct of claim 9, characterized in that is replication-deficient.

11. Vector construct of any of the preceding claims for the use in human or veterinary medicine.

12. Use of the vector construct of any of claims 1 to 10 for the manufacture of a medicament/diagnostic agent for the treatment/diagnosis of tumor diseases, wherein the treatment is carried out prior to or simultaneously with a radionuclide therapy, in particular with iodine-131 or astate-211.

13. Use of two or more vector constructs for the manufacture of a medicament/diagnostic agent for the treatment/diagnosis of tumor diseases, wherein the treatment is carried out prior to or simultaneously with a radionuclide therapy, in particular with iodine-131 or astate-211, characterized in that the two or more vector constructs each contain vector DNA including regulatory sequences and, in different constructs, the NIS gene encoding the sodium/iodide symporter, the TPO gene encoding the thyroid peroxidase, and optionally the TG gene encoding the human thyreoglobulin or any of its sub-units, wherein the two or more vector constructs are optionally included in liposomes, wherein the liposomes may exhibit membrane-bound antibodies, in particular monoclonal antibodies, or other proteins, in particular receptor ligands, specific for tumor surface structures, or are included in adenoviral, retroviral, or other viral vectors of human or animal pathogenicity, wherein the construct may exhibit on its surface natural or artificial tissue-specific, membrane-bound proteins (e.g., fiber proteins), or receptor ligands specific for tumor surface structures, or are included in both

14. Use of claim 12 or 13, wherein the radionuclide therapy is performed with At-211 (as At- or as anionic compound, in particular as AtO⁻, AtO₃⁻, AtO₄⁻, AtO₆⁵⁻), with rhenium-188 and rhenium-186 (as anionic compound) or with yttrium-90 (as anionic compound).

15. Use of any of claims 12 to 14, characterized in that the medicament/diagnostic agent is administered via the intravenous, intraperitoneal, intrathecal, intracranial, intrathoracal, endobronchial, endolymphatic, intraarterial, or intratumoral route.

16. Use of any of claims 12 to 15, characterized in that the tumor disease is a dedifferentiated and medullar thyroid carcinoma, a stomach or intestine tumor, a liver, brain, bone, muscle, kidney, bladder, mylohyoid, uterus, lung, or gonadal tumor, a skin tumor, or a tumor of the exocrine and endocrine glands.

17. Liposome, characterized in that it comprises one or more of the vector constructs of any of claims 1 to 9.

18. The liposome of claim 17, characterized in that it harbors in its membrane antibodies, in particular monoclonal antibodies, or proteins, in particular receptor ligands, specific for tumor cell surface structures.

19. Recombinant adenoviral, retroviral or other virus of human or animal pathogenicity, characterized in that it comprises one or more of the vector constructs of any of claims 1 to 9.
